(19) United States
(12) Patent Application Publication
(10) Pub. No.: US 2017/0220620 A1
(43) Pub. Date: Aug. 3, 2017

(54) SYSTEM AND METHOD FOR SPORTS INFORMATION TRACKING

(71) Applicant: Ali Ahmed ALZAHRANI, Baltimore, MD (US)

(72) Inventor: Ali Ahmed ALZAHRANI, Baltimore, MD (US)

(21) Appl. No.: 15/014,658

(22) Filed: Feb. 3, 2016

(51) Int. Cl. G06F 17/30; G06F 19/00

(52) U.S. Cl. CPC G06F 17/30368; G06F 17/30867; G06F 19/3431; G06F 19/3406; G06F 19/3487

(57) ABSTRACT

A sports information tracking system can be communicably coupled to a device, such that one or more subscribers can request the device to display a profile, wherein the request corresponds to one of the subscribers and causes the profile associated with the subscriber to display on the device. The profiles can be updated based on subscriber submitted data from other subscribers, data gathered from health sensors and performance sensors, and data mined from social media outlets. The system can allow each subscriber to edit at least part of their associated profile. Additionally, the system can be used by subscribers to search for other subscribers to view profiles associated with the other subscribers. The system can also predict injuries, performance ratings, and game results.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Watching sports is a popular past time. It has become so popular that entire television stations, radio stations, and websites are solely devoted to airing and reporting on sporting events. To provide additional content, statistics are tracked for each sport, each team, each player, each coach, etc. The statistics have become a vital part of the content. Statistics can be used to compare teams or individual athletes, evaluate performances, determine when records are broken, and the like.

### SUMMARY

The foregoing paragraphs have been provided by way of general introduction, and are not intended to limit the scope of the following claims. The described embodiments, together with further advantages, will be best understood by reference to the following detailed description taken in conjunction with the accompanying drawings.

A sports information tracking system can be communicably coupled to a device, such that one or more subscribers can request the device to display a profile, wherein the request corresponds to one of the subscribers and causes the profile associated with the subscriber to display on the device. The profiles can be updated based on subscriber submitted data from other subscribers, data gathered from health sensors and performance sensors, and data mined from social media outlets. The system can allow each subscriber to edit at least part of their associated profile. Additionally, the system can be used by subscribers to search for other subscribers to view profiles associated with the other subscribers. The system can also predict injuries, performance ratings, and game results.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 depicts an exemplary overview of the sports information tracking system according to one or more embodiments of the present disclosure.

FIG. 2A depicts an exemplary subscriber view displayed on a device according one or more embodiments of the present disclosure.

FIG. 2B depicts an exemplary automated alert according to one or more embodiments of the present disclosure.

FIG. 3 depicts a swim lane diagram illustrating an exemplary method of displaying a subscriber view.

FIG. 4 depicts an exemplary risk profile according to one or more embodiments of the present disclosure.

FIG. 5 depicts an exemplary hardware description for a server.

FIG. 6 is a flowchart depicting an exemplary method of predicting injuries, performance ratings, and game results.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed subject matter. However, it will be apparent to those skilled in the art that embodiments may be practiced without these specific details. In some instances, well-known structures and components may be shown in block diagram form in order to avoid obscuring the concepts of the disclosed subject matter.

Reference throughout the specification to “one embodiment” or “an embodiment” means that a particular feature, structure, characteristic, operation, or function described in connection with an embodiment is included in at least one embodiment of the disclosed subject matter. Thus, any appearance of the phrases “in one embodiment” or “in an embodiment” in the specification is not necessarily referring to the same embodiment. Further, the particular features, structures, characteristics, operations, or functions may be combined in any suitable manner in one or more embodiments. Further, it is intended that embodiments of the disclosed subject matter can and do cover modifications and variations of the described embodiments.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein, merely describe points of reference and do not necessarily limit embodiments of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit embodiments of the disclosed subject matter to any particular configuration or orientation.

Referring now to the drawings, wherein like reference numerals designate identical or corresponding parts throughout the several views.

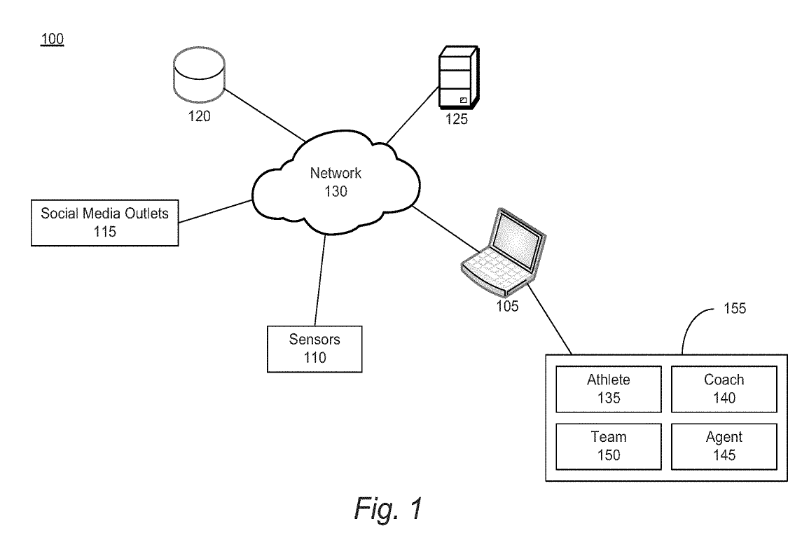

FIG. 1 depicts a sports information tracking system 100 (herein referred to as the system 100). The system 100 can receive various data to determine a level of risk of an athlete, a performance rating, and predict an outcome of a match based on one or more of the risk level and performance rating associated with the athlete or team. The data can be gathered from hardware such as fitness tracking devices, and the data can be mined from social media outlets, for example. The system 100 can include a device 105, at least one sensor 110, social media outlets 115, at least one database 120, at least one server 125, and at least one network 130.

The device 105 can be a smartphone, a computer, a laptop, a tablet, a PDA, and the like. The device 105 can display content for subscribers 155. The subscribers 155 can include an athlete 135, a coach 140, an agent 145, and a team 150. The device 105 can display content, such that the content can be uniquely associated with each of the subscribers 155. Additionally, some of the displayed content included by the subscriber 155 may have limited viewing access. For example, the coach 140 may have access to content in the athlete's profile that another subscriber 155 may not have access to.

The athlete 135 can be the subscriber 155, such that the subscriber 155 can create a profile associated with the athlete 135. The athlete 135, for example, can view other athletes, coaches, teams, and agents, as well as update and control the data associated with the athlete 135 that will be viewed when the content associated with the athlete 135 is requested, via the device 105, for example. In other words, each subscriber 155 can create a profile via any device 105 that can be accessed via any device 105, with predetermined login credentials, for example. The predetermined login credentials can be required to edit the profile of the subscriber 155. However, any device 105 can request the content associated with any subscriber 155 to be displayed via the device 105.

Similarly, the coach 140, the team 150, and the agent 145 can be subscribers 155, such that each subscriber 155 can create a profile that can be viewed when the associated content is requested, as well as request content associated with any subscriber 155 to be displayed via the device 105.

The sensors 110 can include health sensors and performance sensors. Health sensors can include a heartrate monitor, temperature sensors, biosensors for testing blood glucose, cholesterol, drug abuse, and infectious disease, a blood pressure sensor, and the like. The heartrate monitor, for example, can measure heart rate before, during, and after activity. Performance sensors, which may also be able to monitor heartrate, can be used to track data including distance covered, movement, speed, calories burned, and the like, via a FitBit™ or Apple Watch™, for example. The sensors 110 can be wireless connected to a network 130. The sensors 110 can be synced by the athlete via the athlete's personal device, such as a laptop and/or smartphone, for example. Additionally, the data from the sensors 110 can be synced at predetermined times via the network 130 through a wireless internet connection and/or a cellular network.

The social media outlets 115 can include Twitter, Facebook, and the like, such that an associated respective API can be used for data mining as would be understood by one of ordinary skill in the art. The social media outlets 115 can be used to gather data via the APIs from fans, journalists, sports personalities, athletes, and the like sharing on social media. The data from the social media outlets 115 can be analyzed to determine overall trends, gather information on player ratings, determine public opinion of players, coaches, and teams, and the like.

The database 120 can store various information corresponding to the subscribers 155, the sensors 110, and the social media outlets 115. For example, the database 120 can store the profile made by the subscriber 155 such that the appropriate profile of the subscriber 155 can be displayed when requested by the device 105. Similarly, the database 120 can store health data and performance data from the sensors 110, such that the data from the sensors 110 can be associated with one subscriber 155. The results of data mining various social media outlets 115 can also be stored in the database 120 and stored such that the data is associated with a specific subscriber 155.

The server 125 can receive signals from the device 105 via the network 130 causing the server 125 to transmit information to be displayed on the device 105 corresponding to the requested signal. Additionally, the server 125 can receive data from the sensors 110, data from the social media outlets 115, and data stored in the database 120. Further, the server 125 can facilitate various processing for the system 100 as further described herein.

The network 130 can be a public network, such as the Internet, or a private network such as an LAN or WAN network, or any combination thereof and can also include PSTN or ISDN sub-networks. The network 130 can also be wired, such as an Ethernet network, or can be wireless such as a cellular network including EDGE, 3G and 4G wireless cellular systems. The wireless network can also be WiFi, Bluetooth, or any other wireless form of communication that is known.

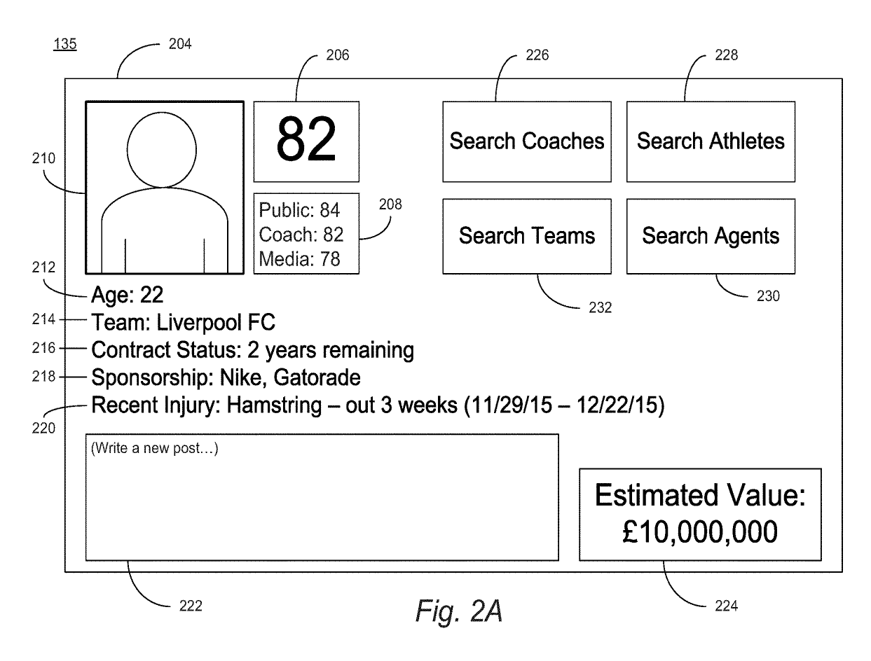

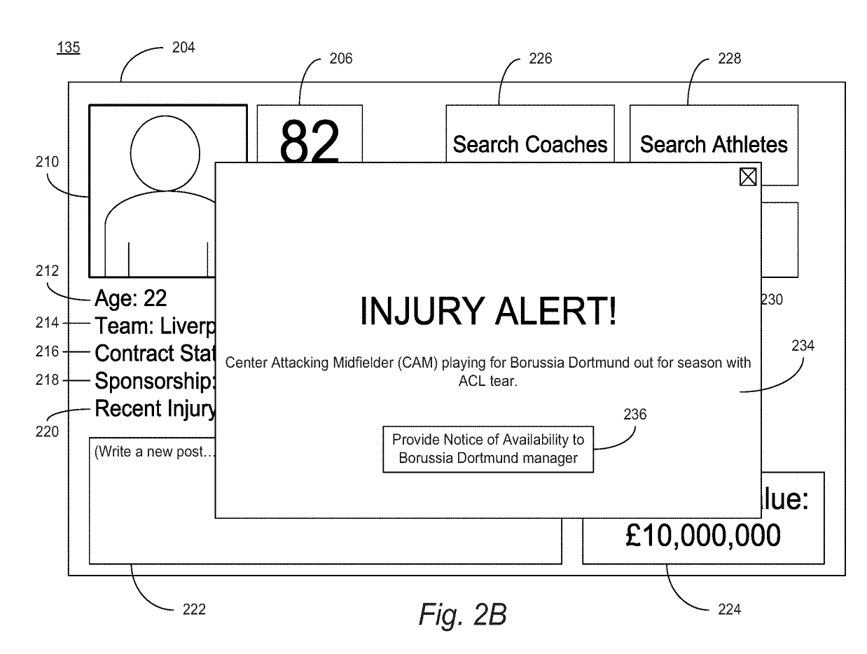

FIG. 2A depicts an exemplary view of a profile 204 for the athlete 135. It should be appreciated that the profile 204 can be adapted to be the profile 204 of any subscriber 155. The athlete's profile 204 can be displayed on the device 105. The profile 204 can include a profile image 210, an overall rating 206, and performance ratings 208 from data gathered from the public, a coach's rating, and a rating determined by the media. The performance ratings 208 can be determined at least via the information provided by the subscribers 155 and/or data mining the social media outlets 115. The profile 204 can also include an age 212 of the subscriber 155, a team 214 with which the subscriber 155 is affiliated, a contract status 216, a sponsorship 218, and a recent injury 220. Additionally, the profile 204 can include posting section 222 in which the subscriber 155 can share words, images, and the like with any device 105 that has requested to view the profile 204. The profile 204 can also include an estimated value 224 of the subscriber 155 associated with the profile 204. Further, the profile 204 can provide search tools such that the subscriber 155 can interact with at least a search coaches section 226, a search athletes section 228, a search teams section 232, and a search agents section 230. Similarly, the profile 204 of any coach 140, agent 145, or team 150 can search for subscribers 155 to view the corresponding profile 204, thereby allowing coaches 140 to search for new athletes 135 to recruit, or agents 145 to search for new teams 150 or athletes 135 to represent, for example.

It should be appreciated that the profile 204 can accommodate any subscriber 155, and the use of the athlete 135 to illustrate the profile 204 is merely exemplary. Similarly, it should be appreciated that each section of the profile 204, such as the team section 214 illustrated as “Liverpool FC”, for example, is merely exemplary and may contain any text, numbers, and/or images to display intended information to any device 105 requesting to view the profile 204.

FIG. 2B depicts an exemplary profile 204 including an injury alert 234. When the server 125 receives information relating to an injury of one of the subscribers 155, for example, the sever 125 can automatically cause the device 105 to display and/or open a smart phone application to alert one or more of the subscribers 155, such as the athlete 135, that an injury has occurred and a position may be available. The injury alert 234 can include a button 236 which can provide a response to the appropriate subscriber 155, such as the coach 140, who may be in need of the athlete 135. The illustrated text is intended to be exemplary and may include any text, numbers, and/or images to display the intended information to any device 105 on which the injury alert 234 has been caused by the server 125 to display. The alert can be initiated based on the data mining of the social media outlets 115 and/or the alert can be received via entry into the profile 204, for example.

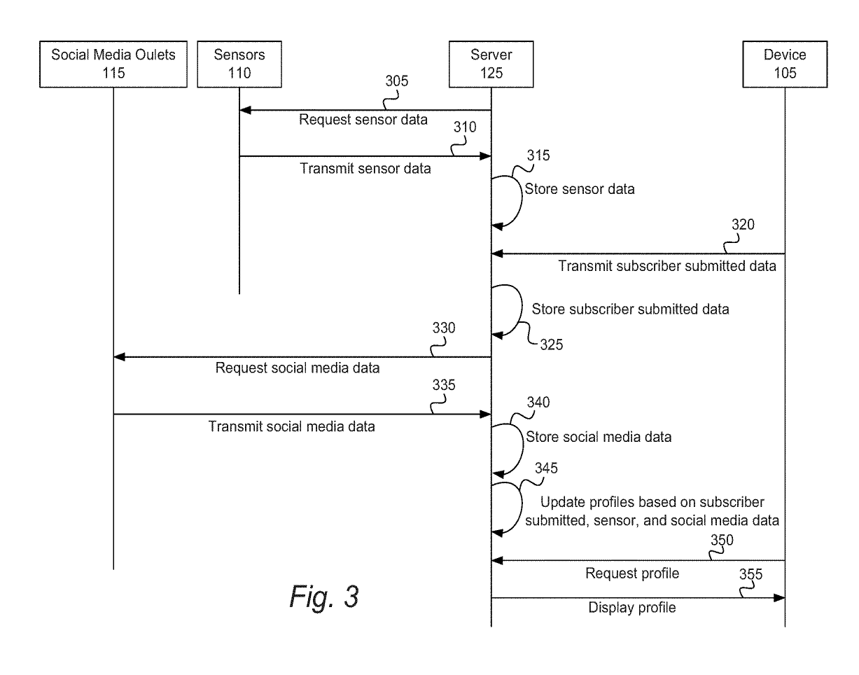

FIG. 3 depicts a swim lane diagram depicting an exemplary method for displaying the profile 204 on the device 105, such that the profile 204 includes the correct content based on the request to display the profile 204 on the device 105.

The server 125, which facilitates the processing for the system 100, can be communicably coupled to the device 105, the sensors 110, and the social media outlets 115 via the network 130.

In step 305, the sever 125 can request sensor data, such as the health data and the performance data, from the sensors 110. The request can occur manually or automatically at predetermined times.

In step 310, the sensor data can be transmitted from the sensors 110 to the server 125.

In step 315, the server 125 can cause the sensor data to be stored. The sensor data can be stored locally in the server 125 or in the database 120, for example.

In step 320, the device 105 can transmit subscriber submitted data to the server 125. For example, when the subscriber 155 updates the profile 204, such as when the coach 140 adds information relating to an injury for the athlete 135, for example, the device 105 can transmit the information associated with the profile 204 to the server 125. The server 125 can then cause the subscriber submitted data to be stored in step 325.

In step 330, the server 125 can request social media data from the social media outlets 115 via corresponding APIs, for example, which can mine the data the server 125 has requested, as would be known to one of ordinary skill in the art.

In step 335, the social media outlets 115 can transmit the requested data to the server 125, and the server 125 can cause the social media data to be stored in step 340.

In step 345, the server 125 can update the profile 204 based on the subscriber submitted data, sensor data, and social media data. The profile 204 can include the updated subscriber submitted data, sensor data, and social media data associated with the subscriber 155.

The device 105 can request the profile 204 from the server 125 in step 350. The server 125 can cause the device 105 to display the requested profile 204, such that the profile 204 is displayed containing the most recent subscriber input, sensor data, and social media data.

It should be appreciated that the steps in the swim lane diagram may not occur in the illustrated numerical order. For example, the subscriber input in step 320 may not be transmitted at any predetermined time, thereby allowing the updates to occur in real time.

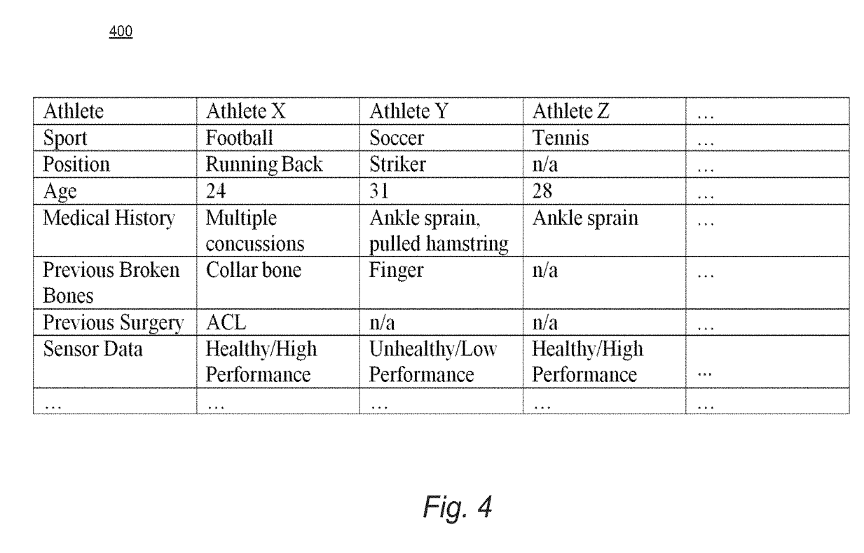

FIG. 4 depicts a risk profile 400, which can optionally be incorporated into the profile 204, according to one or more embodiments of the present disclosure. The risk profile 400 can include information relating to the athletes 135 (e.g., sport, position, age, medical history, previously broken bones, previous surgery, sensor data, etc.), such that the information can be weighted to determine a level of risk associated with the athlete 135. The weighting of the information can be based on factors including the risks associated with a sport, the age of the athlete 135, medical history, and the like. For example, the risk associated with tennis can be considered lower than the risk associated with football as tennis is a non-contact sport. Predetermined risk weights can be applied to the information included in the risk profile. For example, Athlete Y may have previously broken a finger. However, because Athlete Y plays soccer and is not a goalie, Athlete Y may almost exclusively be using their feet. Therefore, the previously broken finger may have a very low risk weight. However, Athlete X may have a medical history revealing multiple concussions, which due to the nature of the sport of football, may have a high risk weight. Additionally, the data from the sensors 110 can be of value to the prediction because the health sensors and performance sensors can provide the most recent data in real time. Therefore, the risk profile 400 can be used to analyze the athlete 135 to determine if the risk associated with the athlete 135 is above a predetermined level of risk. The risk associated with the athlete 135 being above a predetermined level of risk may indicate a high probability of injury, for example. Although the risk profile 400 is illustrated as a table, it should be appreciated that the data being in a table format is merely exemplary, and the data can be stored in the server 125, for example, and associated with each athlete 135 using various computer storage techniques as would be known to one of ordinary skill in the art.

The following equation (Eq. 1) may be used to determine a risk associated with the athlete 135 based on the risk profile 400:

ΣF₁W₁+ . . . +FₙWₙ Eq. 1

Eq. 1 can determine a risk level by taking a sum of the factors (F₁ though Fₙ) multiplied by the weight (W₁ through Wₙ) associated with each factor.

For example, the equation for Athlete X from risk profile 400 may be:

ΣFootball(0.3)+RB(0.25)+Concussions(0.2)+Collar Bone(0.02)+ACL(0.1)+Healthy and High Performance(−0.3) Eq. 2

The result of Eq. 2 (where RB is an abbreviation for the position of running back) results in the sum being equal to a risk level of 0.57. The risk level of 0.57 can be out of 1 to determine a percentage of a 57% risk level. Each factor can have a predetermined weight. For example, because football is a contact sport the risk level associated with the sport can be relatively high, as in 0.3. However, because the sensor data showed that the athlete is healthy and has a high level of performance, the weight can be a negative number so as to counteract the risk associated with the sport, position, age, etc. Although poor health or performance as determined by the sensor data can result in another positive number, thereby increasing the risk level associated with the athlete 135. It should be appreciated that the weights may be adjusted based on severity of a specific factor. For example, 3 concussions may be weight as 0.2, whereas 5 concussions may be weighted as 0.5. Additionally, the weights can be adjusted such that increasing or decreasing the number of factors will not allow the maximum possible risk level to exceed 1 (100%). Also, when the number of factors in the equation changes, the factors that were in the previous equation can be scaled proportionally so as not to alter the risk level associated with each factor.

The threshold can be a predetermined percentage, such as 60%, for example, which can determine that the athlete 135 has a high enough risk level that an injury is likely. The predetermined percentage can be based on the sport. For example, as football is a contact sport, injury is generally accepted as being more common than a non-contact sport, such as tennis, for example. Therefore, the threshold for football may be 60%, whereas the threshold for tennis may be 80%.

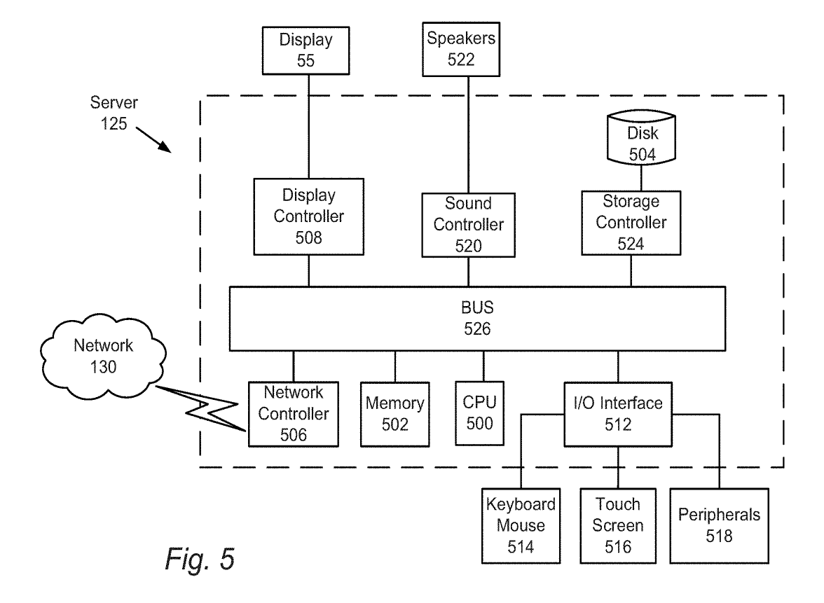

Next, a hardware description of the server 125 according to exemplary embodiments is described with reference to FIG. 5. In FIG. 5, the server 125 includes a CPU 500 which performs the processes described above/below. The process data and instructions may be stored in memory 502. These processes and instructions may also be stored on a storage medium disk 504 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the server 125 communicates, such as a server or computer.

Further, the claimed advancements may be provided as a utility application, background daemon, or component of an operating system, or combination thereof, executing in conjunction with CPU 500 and an operating system such as Microsoft Windows 7, UNIX, Solaris, LINUX, Apple MAC-OS and other systems known to those skilled in the art.

The hardware elements in order to achieve the server 125 may be realized by various circuitry elements, known to those skilled in the art. For example, CPU 500 may be a Xenon or Core processor from Intel of America or an Opteron processor from AMD of America, or may be other processor types that would be recognized by one of ordinary skill in the art. Alternatively, the CPU 500 may be implemented on an FPGA, ASIC, PLD or using discrete logic circuits, as one of ordinary skill in the art would recognize. Further, CPU 500 may be implemented as multiple processors cooperatively working in parallel to perform the instructions of the inventive processes described above.

The server 125 in FIG. 5 also includes a network controller 506, such as an Intel Ethernet PRO network interface card from Intel Corporation of America, for interfacing with network 130. As can be appreciated, the network 130 can be a public network, such as the Internet, or a private network such as an LAN or WAN network, or any combination thereof and can also include PSTN or ISDN sub-networks. The network 130 can also be wired, such as an Ethernet network, or can be wireless such as a cellular network including EDGE, 3G and 4G wireless cellular systems. The wireless network can also be WiFi, Bluetooth, or any other wireless form of communication that is known.

The server 125 further includes a display controller 508, such as a NVIDIA GeForce GTX or Quadro graphics adaptor from NVIDIA Corporation of America for interfacing with display 510, such as a Hewlett Packard HPL2445w LCD monitor. A general purpose I/O interface 512 interfaces with a keyboard and/or mouse 514 as well as a touch screen panel 516 on or separate from display 510. General purpose I/O interface also connects to a variety of peripherals 518 including printers and scanners, such as an OfficeJet or DeskJet from Hewlett Packard.

A sound controller 520 is also provided in the server 125, such as Sound Blaster X-Fi Titanium from Creative, to interface with speakers/microphone 522 thereby providing sounds and/or music.

The general purpose storage controller 524 connects the storage medium disk 504 with communication bus 526, which may be an ISA, EISA, VESA, PCI, or similar, for interconnecting all of the components of the server 125. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

It should be appreciated that the hardware description described herein can similarly describe the hardware of the device 105, such that the device 105 can perform the various processing required by the system 100.

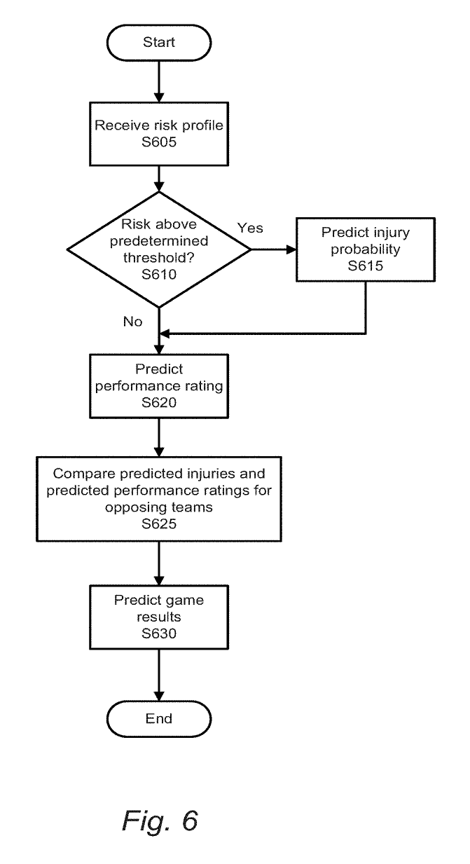

Next, FIG. 6 illustrates an exemplary algorithmic flowchart for predicting injury, performance rating, and game results according to one aspect of the present disclosure.

In S605, the server 125 receives the risk profile 400 from the database 120, such that the database 120 has stored all relevant data for the risk profile 400. The risk profile 400 can determine a level of risk associated with the athlete 135 as described previously herein.

In S610, the server 125 determines if the risk level based on the risk profile 400 is above the predetermined threshold, as described with reference to Eq. 1 and Eq. 2, such that the risk level being above the predetermine threshold may be indicative of a higher probability of injury than if the risk level was below the predetermined threshold. If the risk level is not above the predetermined threshold, then the performance rating is predicted in S620. However, if the risk level is above the predetermined threshold, then there may be a high probability that the athlete 135 may sustain an injury. Once the probability of injury is predicted by the server 125, the performance rating of the athlete 135 may be predicted in S620.

In S620, the server 125 predicts the performance rating of the athlete 135. If the risk level associated is above the predetermine threshold in S610, then the prediction of the performance rating can include the probability of injury. The performance rating prediction can also include information gathered from the sensors 110, the subscriber submitted data from the subscribers 155, the data mined from the social media outlets 115, and the like to determine factors indicative of performance including health, confidence gained from success in recent games/matches, amount of travel required to play the game at an away stadium, and the like, such that the factors indicative of performance can be predetermined and weighted accordingly. The information including performance following success in recent games/matches, amount of travel required, etc. can be determined to have a predetermined weight by analyzing trends associated with those factors. For example, the recorded history and statistics can be analyzed by the server 125 to determine any common trends resulting from the performance resulting from successive victories, the performance resulting from specific away stadiums and the amount of travel required to play, and the like.

In S625, the server 125 can compare the predicted injuries and predicted performance ratings for each player of each of the opposing teams. For example, if a wide receiver of a football team is predicted to have a high performance rating, and the corner back of the opposing football team designated to cover the wide receiver has a high probability of injury and a low predicted performance rating, the comparison of the predicted injuries and performance ratings may result in a favorable comparison for one team over the other team. Additionally, coaches may have an advantage by choosing matchups based on the predicted injuries and predicted performance ratings. The coach may also be more aware of personnel management. For example, if a player has a high likelihood of injury, the coach may want to rest that player to prevent injury. Similarly, if the player has a high predicted performance rating, the coach may want to put that player in the starting line-up, or match that player against a weaker player on the opposing team. The performance ratings calculations based on data discussed herein can cause the app to display the information automatically, similarly and/or at the same time as any injury report causes the app to display the injury alert automatically as in FIG. 2B. Therefore, the game results can be predicted in S630.

In S630, the server 125 can predict the game results, meaning that it may be predicted that one team has a higher probability of winning than the opposing team, for example, based on the predicted injuries and the predicted performance ratings of each athlete of each of the opposing teams. For example, in a football game, a wide receiver with a high predicted performance rating may be matched up against a corner back with a high probability of injury as determined by the server 125. This may present a favorable match up for the team with the healthy wide receiver, and therefore, it may be more likely that the team with the healthy wide receiver will win by taking advantage of the match up. It should be considered that teams and coaches may have performance ratings as well, and the performance ratings of the teams and coaches may be considered in the predicting the game results. After the game results are predicted, the process can end.

An advantage of the system 100 is providing access to information, updated in real-time, between athletes, coaches, teams, and agents. The system provides an overview of extremely large amounts of data, such as the data mined from Twitter, for example, in the form of a public rating. Therefore, an agent 145 or a coach 140, for example, can determine the public's opinion of a player in a single number where sifting through the amount of data necessary to determine the public opinion of a player would not be possible for a human.

Further, the data gathered by the system 100 includes real-time sensor data, thereby providing the most current health and performance data for the subscribers 155 to view when requesting the profile associated with the athlete 135, for example.

Additionally, the automatic injury alert can fast-track the process of replacing an injured athlete. Because the injury alert causes the device to automatically display the profile 204, the coach 140 with the injured athlete can know those athlete's prepared to join the team to replace the injured athlete as soon as possible.

Coaches can make improved decisions for team strategy and personnel choices to maximize the potential of the players, as well as preventing injury. The advantages of the system 100 can also improve the field of sports science and medicine based on the data gathered from the sensors 110 and the risk profile 400, for example.

Having now described embodiments of the disclosed subject matter, it should be apparent to those skilled in the art that the foregoing is merely illustrative and not limiting, having been presented by way of example only. Thus, although particular configurations have been discussed herein, other configurations can also be employed. Numerous modifications and other embodiments (e.g., combinations, rearrangements, etc.) are enabled by the present disclosure and are within the scope of one of ordinary skill in the art and are contemplated as falling within the scope of the disclosed subject matter and any equivalents thereto. Features of the disclosed embodiments can be combined, rearranged, omitted, etc., within the scope of the invention to produce additional embodiments. Furthermore, certain features may sometimes be used to advantage without a corresponding use of other features. Accordingly, Applicant(s) intend(s) to embrace all such alternatives, modifications, equivalents, and variations that are within the spirit and scope of the disclosed subject matter.

1. A system comprising:
a plurality of sensors;
at least one device;
a server;
one or more social media database;
a system database;
a network communicably coupling the device, the system database, the plurality of sensors, the one or more social media databases, and the server, the server including
circuitry configured to
receive sensor data from the plurality of sensors,
receive subscriber submitted data, the data being information added to a profile of the subscriber by the subscriber,
receive social media data from the one or more social media databases,
update subscriber profiles based on the subscriber submitted data, the sensor data, and the social media data,
transmit to the at least one device one or more subscriber profiles when a request is received from the device to view the subscriber profile,
receive an injury alert from at least one of the plurality of sensors, the one or more devices, the one or more social media databases, and the system database, and
automatically transmit to the at least one device the one or more subscriber profiles when receiving an injury alert, the received injury alert causing programming to be activated within the at least one device to cause the injury alert to display on the device.

2. The system of claim 1, wherein the circuitry is further configured to
generate a risk profile,
determine if a risk level calculated as a function of the risk profile is above a predetermined threshold,
predict an injury probability in response to the risk level being above the predetermined threshold, and
predict a performance rating as a function of the injury probability.

3. The system of claim 2, wherein the circuitry is further configured to
compare predicted injury probabilities and predicted performance ratings of opposing teams, and
predict game results based on the comparison of the predicted injury probabilities and the predicted performance ratings of the opposing teams.

4. The system of claim 1, wherein the plurality of sensors include health sensors and performance sensors.

5. The system of claim 1, wherein the subscriber is one of an athlete, a coach, a team, or an agent, each having a corresponding subscriber profile.

6. The system of claim 5, wherein the subscriber profile is updated in real-time.

7. The system of claim 6, further comprising:
calculating an overall rating of the subscriber and associating the overall rating with the subscriber profile.

8. The system of claim 7, wherein the overall rating of the subscriber is calculated as a function of averaging a public rating, a rating from a coach associated with the subscriber, and a media rating.

9. The system of claim 8, wherein the public rating is generated by analyzing the social media data.

10. The system of claim 8, wherein the overall rating of the coach is calculated further based on the subscriber submitted data associated with the coach's subscriber profile.

11. The system of claim 1, wherein the device includes a computer, a laptop, a smart phone, a tablet, and a PDA.

12. The system of claim 3, wherein the risk profile includes a plurality of variables including at least one of the athlete, sport, position, age, medical history, previously broken bones, previous surgery, and sensor data, each of the plurality of variables in the risk profile being assigned a predetermined weight based on a risk associated with the variable.

13. The system of claim 3, wherein the circuitry is configured to automatically transmit the one or more subscriber profiles when receiving the predicted injury probabilities and the predicted performance ratings, wherein the received injury probabilities and the predicted performance ratings cause programming to be activated within the at least one device to cause the predicted injury probabilities and the predicted performance ratings to display on the at least one device.

14. A method of tracking sports information comprising:
receiving sensor data from a plurality of sensors;
receiving subscriber submitted data, the data being information added to a profile of the subscriber by the subscriber;
receiving social media data from one or more social media databases;
updating, via processing circuitry, subscriber profiles based on the subscriber submitted data, the sensor data, and the social media data;
transmitting to a device one or more subscriber profiles when a request is received from the device to view the subscriber profile;
receiving an injury alert from one or more of the plurality of sensors, the one or more devices, the one or more social media databases, and the system database; and
automatically transmitting to the device the one or more subscriber profiles when receiving the injury alert, wherein the received injury alert causing programming to be activated within the device to cause the injury alert to display on the device.

15. The method of claim 14, further comprising:
generating a risk profile;
determining if a risk level calculated as a function of the risk profile is above a predetermined threshold;
predicting an injury probability in response to the risk level being above the predetermined threshold; and
predicting a performance rating as a function of the injury probability.

16. The method of claim 15, further comprising:
comparing predicted injury probabilities and predicted performance ratings of opposing teams; and
predicting game results based on the comparison of the predicted injury probabilities and the predicted performance ratings of the opposing teams.

17. A server comprising:
circuitry configured to
receive sensor data from a plurality of sensors,
receive subscriber submitted data, the data being information added to a profile of the subscriber by the subscriber,
receive social media data from one or more social media databases,
update subscriber profiles based on the subscriber submitted data, the sensor data, and the social media data,
transmit to at least one device one or more subscriber profiles when a request is received from the device to view the subscriber profile,
receive an injury alert from at least one of the plurality of sensors, the one or more devices, the one or more social media databases, and the system database, and
automatically transmit to the at least one device the one or more subscriber profiles when receiving an injury alert, the received injury alert causing programming to be activated within the at least one device to cause the injury alert to display on the device.

18. The server of claim 17, wherein the circuitry is further configured to
generate a risk profile,
determine if a risk level calculated as a function of the risk profile is above a predetermined threshold,
predict an injury probability in response to the risk level being above the predetermined threshold, and
predict a performance rating as a function of the injury probability.

19. The server of claim 18, wherein the circuitry is further configured to
compare predicted injury probabilities and predicted performance ratings of opposing teams, and
predict game results based on the comparison of the predicted injury probabilities and the predicted performance ratings of the opposing teams.
